(19) United States
(12) Patent Application Publication
(10) Pub. No.: US 2024/0302344 A1
(43) Pub. Date: Sep. 12, 2024

(54) NOVEL METHODS AND RELATED SYSTEMS TO ESTIMATE WATER CONTENT OF SUBTERRANEAN CORE SAMPLES

(71) Applicant: CORE LABORATORIES LP, Houston, TX (US)

(72) Inventors: Ajayendra KUMAR, Missouri City, TX (US); Richard FYFE, Tomball, TX (US); Ankit MITTAL, Houston, TX (US); Louis ARGUELLO, Houston, TX (US)

(73) Assignee: Core Laboratories LP, Houston, TX (US)

(21) Appl. No.: 18/284,059

(22) PCT Filed: May 4, 2022
(86) PCT No.: PCT/US2022/027699

§ 371 Date: Sep. 25, 2023

Related U.S. Application Data
(60) Provisional application No. 63/166,779, filed on Mar. 26, 2021.

(51) Int. Cl. G01N 33/24; G01N 1/28; G01N 1/40

(52) U.S. Cl. CPC G01N 33/246; G01N 1/286; G01N 1/4077; G01N 2001/2866

(57) ABSTRACT

A method of processing a core sample includes the steps of: hermetically sealing the core sample in a chamber of an enclosure, comminuting the core sample while the chamber is hermetically sealed, injecting a hydrophilic agent into the chamber, wherein a slurry is formed by at least the hydrophilic agent and water released by the core sample, extracting at least a portion of the slurry from the chamber, separating a fluid from the slurry, analyzing the separated fluid to estimate an amount of water in the fluid, and estimating a water content of the core sample using the estimated amount of water in the fluid.

### BACKGROUND OF THE DISCLOSURE

### 1. Field of Disclosure

The present disclosure relates to processes and methods for estimating water content of samples retrieved from a subsurface formation.

### 2. Description of the Related Art

The costs to construct hydrocarbon producing wells may easily exceed tens of millions of dollars. Therefore, well owners typically seek to characterize a potential hydrocarbon reservoir residing in a subterranean formation as accurately as possible before committing funds and other resources to drill a wellbore and construct an oil well to recover oil and gas from that reservoir. One technique for evaluating subterranean formations involves taking core samples. The core samples can be evaluated to ascertain the geological make-up of a formation and to predict whether the reservoir in that formation could produce hydrocarbons at a quantity and/or rate that justifies the construction of an oil or gas well.

In aspects, the present disclosure addresses the need for enhanced methods and processes for analyzing subsurface core samples used to characterize a subterranean formation and resident hydrocarbon formations.

### SUMMARY OF THE DISCLOSURE

In aspects, the present disclosure provides a method of processing a core sample. The process may include the steps of hermetically sealing the core sample in a chamber of an enclosure; comminuting the core sample while the chamber is hermetically sealed; injecting a hydrophilic agent into the chamber, wherein a slurry is formed by at least the hydrophilic agent and water released by the core sample; extracting at least a portion of the slurry from the chamber; separating a fluid from the slurry; analyzing the separated fluid to estimate an amount of water in the fluid; and estimating a water content of the core sample using the estimated amount of water in the fluid.

In aspects, the present disclosure also provides an apparatus for processing a core sample. The apparatus may include: an enclosure having a chamber, the enclosure being configured to hermetically seal the core sample in the chamber; a comminuting element disposed inside the enclosure and configured to comminute the core sample inside the hermetically sealed chamber while the enclosure is agitated; an injector in selective fluid communication with the chamber, the injector being configured to inject a hydrophilic agent into the hermetically sealed chamber, wherein a slurry is formed by at least the hydrophilic agent and water released by the core sample; and an extractor in selective fluid communication with the chamber, the extractor being configured to extract at least a portion of a slurry from the chamber, wherein the slurry is formed by at least the hydrophilic agent and water released by the core sample. In embodiments, the apparatus may also include a pump in selective fluid communication with the chamber, the pump being configured to reduce a pressure in the chamber to at least below an ambient pressure external to the enclosure.

In aspects, the present disclosure also provides a system for estimating a water content of the core sample. The system may include: an enclosure having a chamber, the enclosure being configured to hermetically seal the core sample in the chamber; a comminuting element disposed inside the enclosure and configured to comminute the core sample inside the hermetically sealed chamber while the enclosure is agitated; an injector in selective fluid communication with the chamber, the injector being configured to inject a hydrophilic agent into the hermetically sealed chamber, wherein a slurry is formed by at least the hydrophilic agent and water released by the core sample; and an extractor in selective fluid communication with the chamber, the extractor being configured to extract at least a portion of a slurry from the chamber, wherein the slurry is formed by at least the hydrophilic agent and water released by the core sample; a separator configured to separate a fluid from the slurry; a fluid analyzer configured to estimate an amount of water in the fluid by analyzing the separated fluid; and a processor configured to estimate a water content of the core sample using the estimated amount of water in the fluid. In embodiments, the system may also include a pump in selective fluid communication with the chamber, the pump being configured to reduce a pressure in the chamber to at least below an ambient pressure external to the enclosure.

The above-recited examples of features of the disclosure have been summarized rather broadly in order that the detailed description thereof that follows may be better understood, and in order that the contributions to the art may be appreciated. There are, of course, additional features of the disclosure that will be described hereinafter and which will form the subject of the claims appended hereto.

### BRIEF DESCRIPTION OF THE DRAWINGS

For detailed understanding of the present disclosure, references should be made to the following detailed description of the preferred embodiment, taken in conjunction with the accompanying drawings, in which like elements have been given like numerals and wherein:

FIG. 1 is a flow chart of one illustrative process in accordance with the present disclosure; and

FIGS. 2A-C are schematic view of a sample processor according to one embodiment of the present disclosure.

### DETAILED DESCRIPTION OF THE DISCLOSURE

As will become apparent below, the present disclosure provides for water content analysis of samples retrieved from subterranean formations. The present disclosure is susceptible to embodiments of different forms. These are shown in the drawings, and herein will be described in detail, specific embodiments of the present disclosure with the understanding that the present disclosure is to be considered an exemplification of the principles of the disclosure, and is not intended to limit the disclosure to that illustrated and described herein. Further, while embodiments may be described as a system made up of several components or as a combination of two or more features, it should be understood that the individual components or individual features may themselves represent advancements over the prior art and may be separately utilized for any given system or combination.

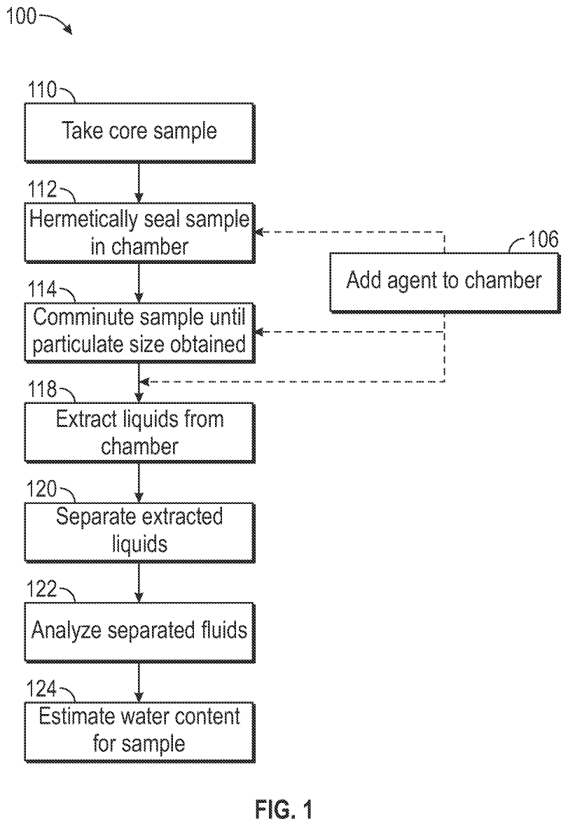

Referring now to FIG. 1, there is shown one illustrative process 100 that may be used to estimate water content in a core sample taken from a subterranean formation of interest. At step 110, a core sample may be recovered from the subterranean formation. This retrieval may be performed by using a coring tool or other device that is conveyed into a borehole intersecting the subterranean formation. Such coring tools may be conveyed via wireline, drill pipe or coiled tubing. The core sample may be preserved, sealed in a container, and transported to a suitable facility for further processing and analysis. In embodiments, the sample may be from an “unconventional” formation, which is generally a formation that has a permeability that is less than ten millidarcy (mD). Many “unconventional” formations have a permeability between one nano-darcy (nD) and one millidarcy (mD). Gas shale is one non-limiting example of an unconventional formation.

The process 100 uses an estimated amount of a hydrophilic (water-miscible) agent to extract water in the core sample. A non-limiting example of a hydrophilic agent is a solvent and electrovalent compound (e.g., NaCl, KCl, etc.). The estimated amount of the hydrophilic agent may be a pre-calculated or predetermined amount based on some basic properties like weight, volume, etc. and measured while being added to the chamber. Adding the hydrophilic agent to the chamber is a step 106, which may occur at different times and in varied amounts. The hydrophilic agent may be added in its entirety or in part at step 112. Alternatively, the entire estimated amount may be added at steps 114. Still another alternative is to add a portion of the estimated amount at step 112 and remaining amounts at step 114. Yet another alternative is to add portions or the entire amount after step 114 and before step 118.

At step 112, the core sample may be analyzed and sealed inside a chamber. The seal is a hermetic seal that prevents fluids and/or solids from entering or escaping the chamber. The analysis may include weighting the core sample. In some embodiments, the precision of the weight may be at least 1/1000 of a gram. Other analyses may include taking dimensional measurements and estimating porosity using known methods such as GRI or NMR. After sealing the chamber, the air from the chamber may be evacuated by using a vacuum pump. Thus, the chamber may be at a vacuum pressure relative to the ambient pressure outside of the chamber.

At step 114, the core sample is comminuted; i.e., broken into particulates under in a controlled environment and conditions as described below. The core sample may be broken into particulates using mechanical action, vibrations, acoustic signals, or any other suitable methods. For example, blades or rollers may physically act on the core sample. Also, the container may be vibrated, spun, or otherwise agitated. One or more fixed or freely moving comminution elements such as ball bearings may also be disposed inside the chamber during such agitation to assist in breaking up the core sample. It should be noted that the comminution process is performed while the chamber is sealed. Therefore, any fluid released while the core sample is broken up is captured within the chamber. The comminution is performed until the core sample is broken into a desired particulate size, average particulate size, maximum particulate size, or ranges of particulate sizes. Optionally, the chamber may be cooled to remove heat generated while comminuting the core sample. This cooling may assist in maintaining a vacuum pressure in the chamber. If present, the hydrophilic agent mixes with the core sample that is being comminuted. Specifically, the hydrophilic agent that comes in contact with the comminuted rock sample mixes with the water released to form a fluid mixture. The fluid mixture may be referred to as a slurry as it contains solids and liquids. The mixing is performed at least until a homogenized fluid mixture is obtained. Suitable hydrophilic agents include any agent that is miscible with water; e.g., organic solvents with low polarity such as hexanes, toluene, dichloromethane and diethyl ether.

If some or all of the hydrophilic agent was added at step 112 and/or step 114, then comminuting the core sample may provide adequate mixing. If not, a separate mixing step (not shown) may be performed. In this step, while some comminution may take place, the main intent of this step is to form a homogenous slurry. A separate mixing step (not shown) may be used if some or all of the hydrophilic agent is added after step 114.

At step 118, at least a portion of the homogenous fluid mixture is extracted from the container. At step 120, a separation process is performed to separate a fluid, i.e, liquids and gases, from the fines and other solids entrained in the mixture. The separated fluid may have residual, minimal amounts of solids. One non-limiting process is centrifugal separation. At step 122, one or more tests may be performed to collect information that may be used to estimate the amount of water in the separated fluid. One non-limiting test is potentiometric titration. At step 124, the data from the step 122 may be used in conjunction with the step 106 to estimate the water content in the core sample. The data from the step 124 may be used in conjunction with step 112 and some other properties measured like sample weights, dimensions, GRI, etc. to calculate water, hydrocarbon and gas saturations or in bulk volume (cm3 of oil, water and/or gas) in the rock sample.

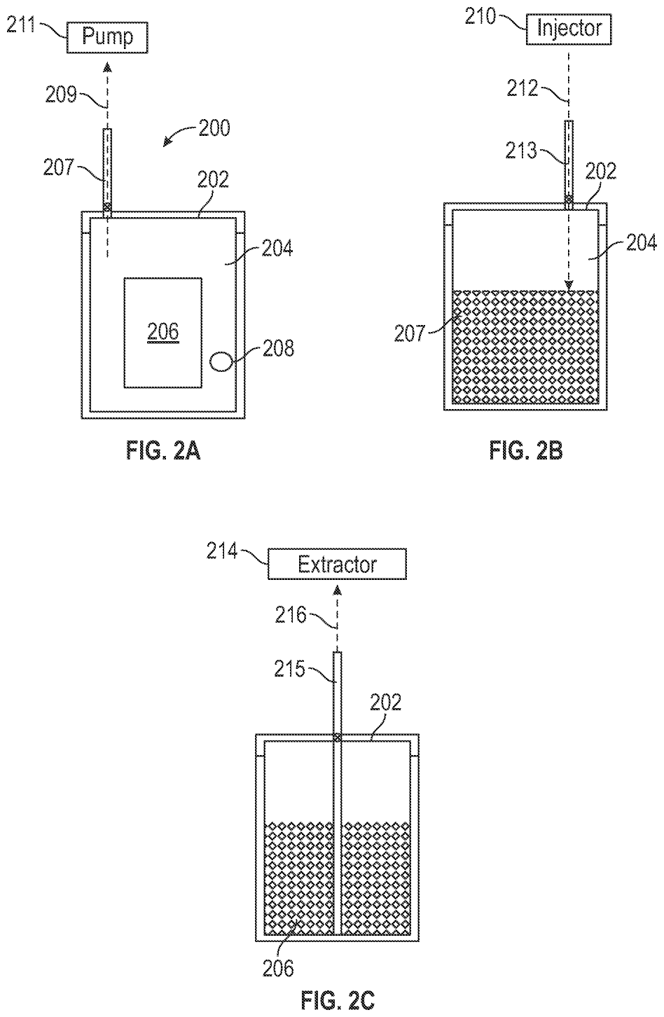

Referring now to FIGS. 2A-C, there is schematically shown a sample processor 200 in accordance with one embodiment of the present disclosure.

FIG. 2A illustrates the configuration of the sample processor used at step 112 (FIG. 1). The sample processor 200 may include an enclosure 202 that has a hermetically sealed chamber 204 in which a core sample 206 is positioned. The enclosure 202 may include sealing elements (not shown) that prevent the entry and escape of fluids (e.g., liquids, gases, vapors, etc.) while the core sample 206 is being processed in the chamber 204. The seals (not shown) are also configured to hold a vacuum pressure relative to ambient pressure external to the enclosure 202. The sample processor 200 may also have one or more comminution elements 208 that are configured to contact and break up the core sample 206. The comminution element 208 may be fixed or free floating. Free floating comminution elements 208 may be activated by vibrating, spinning, or otherwise agitating the enclosure 202. Fixed comminution elements 208 may be activated using a motor or by agitating the enclosure 202. The comminution elements 208 may be beads, fins, serrated edges, blades, rods, rollers, etc. In other embodiments, one or more walls defining the chamber 204 as a comminution element. That is, the enclosure 202 is agitated and the core sample 206 is broken up during contact with the interior walls of defining the chamber 204. To evacuate the chamber 204, the sample processor 200 includes a tube or other conduit 207 through which air 209 in the chamber 204 may be extracted by using a vacuum pump 211 or other suitable device without allowing other fluids to enter the chamber 204. Thus, the vacuum pump 211 is in selective fluid communication with the chamber 204.

FIG. 2B illustrates the sample processor 200 configured to add the hydrophilic agent, which is generally step 106 (FIG. 1). In this configuration, the sample processor 200 may include an injector 210 for injecting a hydrophilic agent 212. The injector 210 may be a fixed or removable component of the sample processor 200 and be configured to introduce the hydrophilic (water-miscible) agent 212 into the chamber 204 via a line 213 without allowing other fluids to enter or escape the chamber 204 during the injection of the hydrophilic agent 212. Thus, the injector 210 is in selective fluid communication with the chamber 204. The injector 210 may be a syringe, pump, or other suitable device configured to dispense a fluid substance into the chamber 204. As shown, the core sample after agitation is broken up and composed of particulates of a desired size or range of sizes. The particulates making up the core sample are labelled with numeral 207. It should be appreciated that any water released during the agitation is trapped in the chamber 204 and becomes mixed with the hydrophilic agent 212. The agitation may be continued until the hydrophilic agent 212 is fully mixed with the water released by the core sample 207.

FIG. 2C illustrates the sample processor 200 configured to extract homogeneous slurry from the chamber 204, which is generally step 118 (FIG. 1). For this extraction, the sample processor 200 may include an extractor 214 for extracting the slurry 216, which is a mixture of water, the hydrophilic agent 212 (FIG. 2B), and entrained solids (not shown). The extractor 214 may be a fixed or removable component of the sample processor 200 and be configured such that no fluids can enter or escape the chamber 204 during the extraction of the slurry 216 via a line 215. Thus, the extractor 214 is in selective fluid communication with the chamber 204. The extractor 214 may be a syringe, pump, or other suitable device configured to extract a slurry from the chamber 204. It should be appreciated that any water released during the agitation is contained in the slurry 216. Thereafter, the mixture 216 may be processed and analyzed as previously discussed.

It should be noted that FIGS. 2A-C illustrate only one non-limiting embodiment of a sample processor according to the present disclosure. For example, in embodiments, a sample analysis unit may integrate the sample processor with a centrifugal separator and a titration analyzer. Also, in embodiments, the pump 211, the injector 210, and extractor 214 may use the same lines or different lines for fluid communication with the chamber 204. And in embodiments, the several steps may be performed manually or be automated.

The foregoing description is directed to particular embodiments of the present disclosure for the purpose of illustration and explanation. It will be apparent, however, to one skilled in the art that many modifications and changes to the embodiment set forth above are possible without departing from the scope of the disclosure. Thus, it is intended that the following claims be interpreted to embrace all such modifications and changes.

What is claimed is:

1. A method of processing a core sample, comprising:
(a) hermetically sealing the core sample in a chamber of an enclosure;
(b) comminuting the core sample while the chamber is hermetically sealed;
(c) injecting a hydrophilic agent into the chamber, wherein a slurry is formed by at least the hydrophilic agent and water released by the core sample;
(d) extracting at least a portion of the slurry from the chamber;
(e) separating a fluid from the slurry;
(f) analyzing the separated fluid to estimate an amount of water in the fluid; and
(g) estimating a water content of the core sample using the estimated amount of water in the fluid.

2. The method of claim 1, wherein at least a portion of the hydrophilic agent is injected before comminuting the core sample.

3. The method of claim 1, wherein at least a portion of the hydrophilic agent is injected during the comminuting of the core sample.

4. The method of claim 1, wherein at least a portion of the hydrophilic agent is injected after the comminuting of the core sample.

5. The method of claim 1, wherein at least a portion of air is evacuated from the chamber before injecting the hydrophilic agent into the chamber.

6. The method of claim 1, wherein the hydrophilic agent is a solvent and electrovalent compound.

7. The method of claim 1, further comprising maintaining a vacuum pressure in the chamber while comminuting the core sample.

8. An apparatus for processing a core sample, comprising:
(a) an enclosure having a chamber, the enclosure being configured to hermetically seal the core sample in the chamber;
(b) a comminuting element disposed inside the enclosure and configured to comminute the core sample inside the hermetically sealed chamber while the enclosure is agitated;
(c) an injector in selective fluid communication with the chamber, the injector configure to inject a hydrophilic agent into the hermetically sealed chamber, wherein a slurry is formed by at least the hydrophilic agent and water released by the core sample; and
(d) an extractor in selective fluid communication with the chamber, the extractor configured to extract at least a portion of a slurry from the chamber, wherein the slurry is formed by at least the hydrophilic agent and water released by the core sample.

9. The apparatus of claim 8, further comprising a pump in selective fluid communication with the chamber, the pump configured to reduce a pressure in the chamber to at least below an ambient pressure external to the enclosure.

10. A system for estimating a water content of the core sample, comprising:
(a) an enclosure having a chamber, the enclosure being configured to hermetically seal the core sample in the chamber;
(b) a comminuting element disposed inside the enclosure and configured to comminute the core sample inside the hermetically sealed chamber while the enclosure is agitated;
(c) an injector in selective fluid communication with the chamber, the injector configured to inject a hydrophilic agent into the hermetically sealed chamber, wherein a slurry is formed by at least the hydrophilic agent and water released by the core sample; and
(d) an extractor in selective fluid communication with the chamber, the extractor configured to extract at least a portion of a slurry from the chamber, wherein the slurry is formed by at least the hydrophilic agent and water released by the core sample.
(e) a separator configured to separate a fluid from the slurry;
(f) a fluid analyzer configured to estimate an amount of water in the fluid by analyzing the separated fluid; and
(g) a processor configured to estimate a water content of the core sample using the estimated amount of water in the fluid.
